(19) United States
(12) Patent Application Publication
(10) Pub. No.: US 2009/0240313 A1
(43) Pub. Date: Sep. 24, 2009

(54) Electrode for a Neuromuscular Stimulator

(75) Inventor: Felix Buhlmann, Lausanne (CH)

Correspondence Address: ROPES & GRAY LLP, PATENT DOCKETING 39/41, ONE INTERNATIONAL PLACE, BOSTON, MA, 02110-2624, US

(73) Assignee: COMPEX MEDICAL S.A., Ecublens (CH)

(21) Appl. No.: 11/920,298

(22) PCT Filed: May 12, 2006
(86) PCT No.: PCT/IB2006/051501

§ 371 Date: May 22, 2009

(30) Foreign Application Priority Data
May 12, 2005 (CH) 00872/05

(51) Int. Cl. A61N 1/04

(52) U.S. Cl. 607/115

(57) ABSTRACT

An electrode for neuromuscular stimulator comprising a conductive layer, for example of metal, on a resistive layer, the lower face of the resistive layer being intended to contact the skin of a user, characterized in that the specific impedance Zᵣ of the resistive layer is at least equal to half of the average impedance Zᵤ of the portion of derma having to contact the lower face of the resistive layer.

### FIELD OF THE INVENTION

The present invention relates to the field of neuromuscular stimulation and more particularly the electrodes used to this end.

### STATE OF THE ART

One knows various types of electrodes which can be used for neuromuscular stimulation.

The muscles or the nerves are stimulated by means of a current which passes through a pair of stimulation electrodes. As an example, one can use electrodes having the dimensions of 5×5 cm² and a current of approximately 120 mA. To obtain this current, it is necessary to apply a voltage of approximately 60 V to 150 V between the two electrodes knowing that one observes a voltage drop of approximately 20 V to 30 V at the level of internal tissues. The remainder, that is 30 V to 130 V, is used by the two electrode-skin systems. The voltage is thus between 15 V and 65 V at the level of each electrode-skin system.

The electrodes of the state of the art present the disadvantage of inducing discomfort sensations for the user, and even of the burns in certain cases (for example use of electrodes for cardiac defibrillation).

These problems originate in the fact that the density of current is not uniform in the zone of contact electrode-skin.

Accordingly, there is a need to be able to rectify this situation.

### SUMMARY OF THE INVENTION

Thus, an aim of the invention is to improve the known stimulation devices.

Another aim of this invention is to reduce, even eliminate, the above mentioned problems due to the absence of uniformity of the density of current.

The invention will be better understood using the following description of embodiments and diagrammatic figure in which

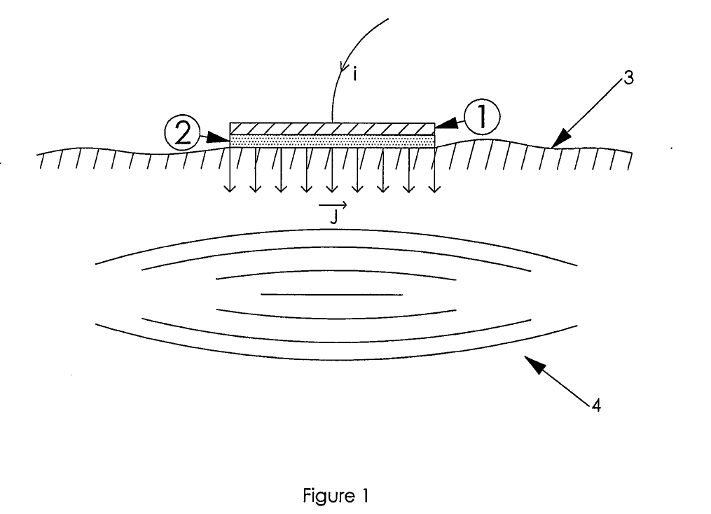

FIG. 1 represents a cross-section of an electrode according to the invention applied on the skin of a patient.

Accordingly the electrode for neuromuscular stimulation comprises a conductive layer 1, for example made of metal, disposed on a resistive layer 2, the lower face of the resistive layer 2 being intended to contact the skin of a user 3. Underneath the electrode, one has in addition represented in a diagrammatic way a muscle 4 intended to be stimulated.

The electrode according to the invention is characterized in that the specific impedance Zᵣ of the resistive layer 2 is at least equal to half the average specific impedance Zᵤ of the portion of derma having to contact the lower face of the resistive layer 2.

In the present application, the concept of “resistive layer” must be understood as any layer which has a certain impedance.

It was indeed observed in a surprising way that a density of current quasi uniform on the level of the electrode-skin interface can be observed if the specific impedance of the resistive layer Zᵣ is at least equal to half the average specific impedance Zᵤ of the portion of derma.

Preferably, the specific impedance Zᵣ of the resistive layer 2 is at least equal to the average specific impedance Zᵤ of the portion of derma having to contact the lower face of the resistive layer 2.

It is appropriate to note that the known electrodes of the state of the art have a specific impedance, typically about 500 Ω cm², which is quite lower than the average specific impedance Zᵤ of the portion of derma having to contact the lower face of the resistive layer.

The use of a low impedance for the electrodes makes it possible to minimize the source of voltage.

It results that with the electrodes of the state of the art, only a small fraction of the voltage generated at the level of the electrode-skin system is used on the electrodes, the greatest fraction of voltage being found at the level of biological tissues, which has as consequence that the distribution of current is mainly determined by the specific impedance of the biological tissues.

Since the latter varies from one point to another of the skin, in particular because of the presence of sudoriferous channels, the variation of its thickness, the invagination around the hairs, its state of cleanliness, its moisture, the perfusion and the peripheral vasodilatation, it results therefrom that the density of current is not uniform at the level of the electrode-skin system.

Within the frame of the present invention, one understands by “average specific impedance Zᵤ of the portion of derma”, the specific impedance seen by the electrode at the level of the electrode-skin interface. It can be evaluated by measuring the density of current under a metallic electrode of the same shape applied directly on the same place of the skin. The average specific impedance Zᵤ of the portion of derma is given as well by the geometry of the electrode-skin system as by the impedances of various tissues.

Preferably, the specific impedance Zᵣ of the resistive layer is at least equal to 2000 Ω cm² for a system comprising electrodes of 25 cm² and which uses a voltage of 10 V at the level of the electrode.

By choosing a value of the specific impedance of the resistive layer Zᵣ sufficiently high, the current at the level of the electrode-skin interface is primarily imposed by the value of the specific impedance of the resistive layer Zᵣ, which makes it possible to obtain a density of current relatively uniform at the level of the electrode-skin system. In other words, more the impedance of the resistive layer will be “dominant” compared to the impedance of the skin, more the density of current will be uniform at the level of the system electrode-skin. With a specific impedance of the resistive layer equal to half of the average specific impedance Zᵤ of the portion of derma having to contact the lower face of the resistive layer 2, the effect of uniformization of the density of current is minimal, while if the ratio is about 10, the effect of uniformization is much more important.

Indeed, when the current is primarily imposed by the impedance of the resistive layer Zᵣ, its value is constant and the density of current uniform because the impedance of the resistive layer Zᵣ is a constant.

On the other hand, if the current was primarily imposed by the average impedance Zᵤ of the portion of derma, as it is the case with the electrodes of the state of the art, its value would be variable, and the density of current non-uniform, because the average impedance Zᵤ is variable. More precisely, each point of the derma being under the electrode is characterized by a specific impedance Zₚ and which generally differs from the impedance of neighbouring points. Thus, when the current is imposed by Zₚ (or Zᵤ), the density of current varies from one point to another of the electrode-skin interface.

According to an advantageous embodiment of the invention, the thickness of the resistive layer of the electrode is less than 2 mm.

It goes without saying the invention is not limited to the examples and values previously indicated, the choice of the specific impedance of the resistive layer being variable according to the envisaged use (muscular stimulation for application in the field of sports, defibrillation, iontophoresis, etc).

It is indeed known that the impedance of the skin is influenced by the frequency of stimulation. During a low frequency stimulation, the skin has a high average specific impedance while during a high frequency stimulation, the average specific impedance of the skin is low. Consequently, one can adapt the specific impedance of the resistive layer, by applying the principles of this invention, with the specific impedance of the skin which itself depends on the frequency of stimulation envisaged.

As example, in the case of the defibrillation, one uses high frequency impulses, with the consequence of a low specific impedance of the portion of derma having to contact the lower face of the resistive layer. As in the case of the defibrillation one often observes the presence of burns on the skin zone close to the electrode edge, the principle of the invention makes it possible to effectively uniformize the density of current and to avoid such burns.

As indicated above, the electrode according to the invention can be used with all types of stimulators, such as for example the one described in U.S. Pat. No. 6,324,432 or with the stimulators of the range marketed under the name Compex® (called MI-SPORT, SPORT ELITE, ENERGY, MI-FITNESS, FULL FITNESS, FITNESS, DUOFIT, VITALITY, BODY), incorporated by reference in the present application.

By applying the principles of the invention, one can also consider the use of capacitive electrodes instead of resistive electrodes. Indeed, what is important it is that it is the impedance of the resistive layer in contact with the skin that dominates in order to dictate and uniformize the density of current, the dominant impedance being resistive or capacitive. Hence, the resistive layer can be made from a capacitive layer.

1. An electrode for neuromuscular stimulation comprising a conductive layer on a resistive layer, a lower face of the resistive layer being intended to contact the skin of a user, wherein a specific impedance Zᵣ of the resistive layer is at least equal to half of an average impedance Zᵤ of a portion of derma having to contact the lower face of the resistive layer.

2. The electrode according to claim 1, wherein the specific impedance Zᵣ of the resistive layer is equal to the average impedance Zᵤ of the portion of derma having to contact the lower face of the resistive layer.

3. The electrode according to claim 1, wherein the specific impedance Zᵣ of the resistive layer is at least equal to 2000 Ω cm² for a system comprising electrodes of 25 cm² and which uses a voltage of 10 V at the level of the electrode.

4. The electrode according to claim 1, wherein a thickness of the resistive layer is equal to, or lower than, 2 mm.

5. The electrode according to claim 1, in which the resistive layer is a capacitive layer.

6. A neuromuscular stimulator comprising at least one electrode according to claim 1.
